(19) United States
(12) Patent Application Publication
(10) Pub. No.: US 2009/0053716 A1
(43) Pub. Date: Feb. 26, 2009

(54) METHOD OF DETECTING HUMAN CYTOCHROME P450 (CYP) 2D6 GENE MUTATION

(76) Inventors: Naoko Nakamura, Kawasaki-shi (JP); Tsuyoshi Fukuda, Suita-shi (JP); Junichi Azuma, Suita-shi (JP); Nobuhiro Gemma, Yokohama-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/175,070

(22) Filed: Jul. 17, 2008

(30) Foreign Application Priority Data
Jul. 18, 2007 (JP) 2007-187493

(51) Int. Cl. C12Q 1/68

(52) U.S. Cl. 435/6

(57) ABSTRACT

A defect or multi-existence of a CYP2D6 gene is detected with a primer includes a complementary sequence to a sequence which is common between the CYP2D6 gene and a CYP2D8 gene but different from a CYP2D7 gene and which contains one or more of bases at the 86-, 90- and 93-positions in Exon 9 region of the CYP2D6 gene.

### CROSS-REFERENCE TO RELATED APPLICATIONS

This application is based upon and claims the benefit of priority from prior Japanese Patent Application No. 2007-187493, filed Jul. 18, 2007, the entire contents of which are incorporated herein by reference.

### BACKGROUND OF THE INVENTION

1. Field of the Invention

The present invention relates to a method of detecting a defect and multi-existence of a human cytochrome P450 (CYP) 2D6 gene.

2. Description of the Related Art

Drug-metabolizing enzymes have been attracting attention as a cause for individual differences in the pharmacokinetics in the living body. Among them, human cytochrome P450 (CYP) 2D6 is one of the most important drug-metabolizing enzymes. CYP2D6 is an enzyme that metabolizes about 20 to 30% of clinically used drugs such as β-blockers, psychotropic drugs, antidepressant, and antiemetic drugs.

In a CYP2D6 gene related to the drug-metabolizing enzyme CYP2D6, there are so many mutant alleles, and 80 or more alleles have been confirmed until now. The efficiency of drug metabolism exhibited by the enzyme varies depending on each mutant allele; for example, there are a mutant allele that reduces the enzyme activity, a mutant allele that completely loses the enzyme activity, and a mutant allele that enhances the metabolic activity due to the presence of multiple copies by multi-existence of the CYP2D6 gene, relative to the wild type (CYP2D6*1).

Examples of mutant alleles that do not have the enzyme activity include CYP2D6*3, *4, *5, *6, *7, *8, *11, *12, *13, *14, *15, *16, *18, *21 and *36. The phenotype of heterozygotes or homozygotes of these alleles is a poor metabolizer (PM).

A mutant allele that reduces the enzyme activity is for example CYP2D6*10, and the phenotype of its homozygote is an intermediate metabolizer (IM). This type appears frequently in Orientals.

The phenotype of CYP2D6*2 though regarded to have a relatively reduced activity is classified, like CYP2D6*1, into an extensive metabolizer (EM).

The phenotype of mutant alleles having multiple copies (2 to 13) of CYP2D6*2 (CYP2D6*1, *35) is an ultrarapid metabolizer (UM) having an enhanced activity.

The frequency of mutant alleles of CYP2D6 varies significantly depending on race. The frequency of PM is low among the Japanese (less than 1%) but high among the Caucasians (7 to 10%). The allele as a cause for PM in Orientals is mainly CYP2D6*5 (5 to 6%) or CYP2D6*14 (2%). The allele as a cause for PM in Caucasians is mainly CYP2D6*4 (23%). The frequency of CYP2D6*5 in Caucasians is 4% which is slightly lower than in Orientals. CYP2D6*10 with a reduced activity appears frequently in Orientals, but is as low as 2.6% in Caucasians (see Droll, K et al., Pharmacogenetics 1998, 8:325-333). A multigene type (CYP2D6*2×N) is lower in Orientals but is abundant in Southern Europe and around North Africa.

In recent years, there is need for examination of the allele type of the CYP2D6 gene in order to select drug dosages and therapeutic methods adapted to individuals. For detecting single nucleotide polymorphism (SNP) and insertion or deletion of several nucleotides in the CYP2D6 gene, it is possible to use detection methods reported until now, for example, the Taq-man method, the invader method, the SSCP method, the PCR-RFLP method, the allele specific primer PCR method, the allele specific oligonucleotide hybridization analysis, and the like.

However, the detection of a CYP2D6 gene defect type (CYP2D6*5) or a CYP2D6 gene multi-existence type (CYP2D6*2×N) is very difficult.

As a method of detecting a CYP2D6 gene defect or multi-existence, the Southern blot method has been carried out for a long time (Skoda et al., Proc. Natl. Acad. Sci. USA, Vol. 85, pp. 5240-5243, 1998). In this method, a CYP2D6 defect or multi-existence is detected from sizes (13 kbp, 29 kbp, 42 kbp, 44 kbp) of bands obtained by treating a DNA with a restriction enzyme XbaI. There is however a problem that this method is very troublesome in operation and requires 2 to 3 days.

As another detection method, a method for PCR amplification of a long region has been reported (see Steen et al., Pharmacogenetics, 5, 215-223, 1995 and Johansson et al., Pharmacogenetics, 6, 351-355, 1996). In this method, the presence of a CYP2D6 gene defect or multi-existence can be examined, but its number cannot be known. That is, whether only one of two alleles possessed by an individual, or both of them, is a defect or multi-existence type cannot be determined.

As still another method, a method of measuring the gene number of CYP2D6 has been reported. The gene number of CYP2D6 is 0 for CYP2D6*5 homozygotes, 1 for CYP2D6*5 and normal-type heterozygotes, 2 for normal-type homozygotes, or 3 for CYP2D6*2×2 and normal-type heterozygotes. Thus, a CYP2D6 gene defect or multi-existence can be detected by determining the gene number of CYP2D6 (0, 1, 2, 3, 3 or more). In this method, a region specific to the CYP2D6 gene is amplified with primers. However, amplification does not occur where the gene number of CYP2D6 is 0. This lack of amplification cannot be distinguished from the lack of amplification attributable to a trouble in a thermal cycler or to an error in operation such as failure to add an amplification reagent.

When the gene number of CYP2D6 is 1, 2, 3, or 3 or more, the amounts of final amplified products in the respective cases become virtually the same and can thus not be distinguished from one another. As a method of solving this problem, a method of introducing a control gene is used. Elke Schaeffeler et al. HUMAN MUTATION 22: 476-485, (2003) have used an albumin gene as a control. The albumin gene is most suitable control because its gene number is always 2. The albumin gene is amplified together with the CYP2D6 gene in the same tube by PCR. The gene number of CYP2D6 is determined by comparing the rate of amplification of the albumin gene with the rate of amplification of the CYP2D6 gene by the Taq-man method. However, this analysis by the Taq-man method is problematic in that an expensive and large instrument consisting of a thermal cycler integrated with a spectrofluorometer is needed.

Erik Soderback et al. Clinical Chemistry 51:3, 522-531 (2005) described that a CYP2D8 gene has also used as the control. The CYP2D8 gene and CYP2D7 gene are pseudogenes with very high homology to CYP2D6. The CYP2D8 gene, similar to the albumin gene, is most suitable control because its gene number is always 2. The CYP2D7 gene, on the other hand, is not suitable for the control because of its possible multi-existence. By utilizing the high homology of the CYP2D8 gene to the CYP2D6 gene, primers are designed toward a region common between the CYP2D8 gene and CYP2D6 gene but different from the CYP2D7 gene, thereby specifically amplifying the CYP2D8 gene and CYP2D6 gene only by the method described above. After amplification, the amount of the amplified product of CYP2D8 and the amount of the amplified product of CYP2D6 are compared with each other by the pyrosequence method, thereby determining the gene number of CYP2D6.

In the mutant alleles of the CYP2D6 gene, however, there is CYP2D6*36 having no enzyme activity. Because this allele has no enzyme activity, it should not be counted among gene number, but when primers designed to correspond to Exon 6 region as described by Erik Soderback et al. (2005) supra are used, its gene number is inevitably counted. This problem is fatal to examination of Orientals having CYP2D6*36 appearing with high frequency. When a mutant allele (CYP2D6*36-CYP2D6*10) having single enzymatically active gene is determined by the Taq-man method, the determined number of the enzymatically active gene lies between 1 and 2 because of the influence of CYP2D6*36, thus making accurate determination unfeasible even by the Taq-man method. The analysis by the pyrosequence method is troublesome in operation and problematic because of the need for a relatively long time in examination.

### BRIEF SUMMARY OF THE INVENTION

According to the present invention, there is provided a method of detecting a defect or multi-existence of a CYP2D6 gene, the method comprising: a step of amplifying a CYP2D6 gene and a CYP2D8 gene with a pair of primers to obtain amplified products; a step of determination of amplified products of the CYP2D6 gene and CYP2D8 gene respectively; and a step of comparing the amount of the amplified product of the CYP2D6 gene with the amount of the amplified product of the CYP2D8 gene, wherein one of the pair of primers comprises a complementary sequence to a sequence which is common between the CYP2D6 gene and CYP2D8 gene but different from a CYP2D7 gene and which contains a part or all of bases at the 86-, 90- and 93-positions in Exon 9 region of the CYP2D6 gene.

In one aspect, the amplified products are determined by detecting detection sequences specific to the amplified product of the CYP2D6 gene and the amplified product of the CYP2D8 gene, respectively. In another aspect, the genes are amplified by the LAMP method. In another aspect, the amplified products are detected with nucleic acid probes complementary to the detection sequences. The nucleic acid probe is preferably immobilized on a substrate.

According to the present invention, the gene number of CYP2D6 can be determined without counting CYP2D6*36 having no enzyme activity, thus enabling accurate detection of a CYP2D6 gene defect or multi-existence.

### BRIEF DESCRIPTION OF THE SEVERAL VIEWS OF THE DRAWING

FIG. 1 is a schematic diagram showing a gene structure of CYP2D6;

FIG. 2 shows a CYP2D7 substitution region located in Exon 9 region of CYP2D6*36;

FIG. 3 shows the genotype of CYP2D6 appearing highly frequently among the Japanese and its mutation site;

FIG. 4 shows nucleotide sequences in the Exon 9 region and its vicinity of CYP2D6, CYP2D7 and CYP2D8;

FIG. 5 is a plan schematic diagram of a probe-immobilized substrate in one embodiment;

FIG. 6 is a plan schematic diagram of a probe-immobilized substrate in another embodiment;

FIG. 7 is a schematic diagram showing the arrangement of LAMP primers;

FIG. 8 shows an intermediate product by the LAMP method and the positions to which inner primers (FIP, BIP) anneal;

FIG. 9 shows arrangement of loop primers (LFc, LBc);

FIG. 10 shows an intermediate product by the LAMP method and the positions to which loop primers (LBc, LBc) anneal;

FIG. 11 shows arrangement of detection sequences (FP, FPc, BP and BPc);

FIG. 12 shows LAMP primers and detection sequences in one embodiment;

FIG. 13 shows detection not using block nucleic acids;

FIG. 14 shows the results of Southern blot analysis (XbaI-RFLP);

FIG. 15 shows the results of Southern blot analysis (EcoRI-RFLP);

FIG. 16 shows the results of PCR-RFLP analysis;

FIG. 17 shows the results of nested PCR analysis;

FIG. 18 shows design regions of LAMP primers and detection sequences used in the Example;

FIG. 19 shows the results in the Example; and

FIG. 20 is an analytic plot of results of detection of 19 Japanese samples.

### DETAILED DESCRIPTION OF THE INVENTION

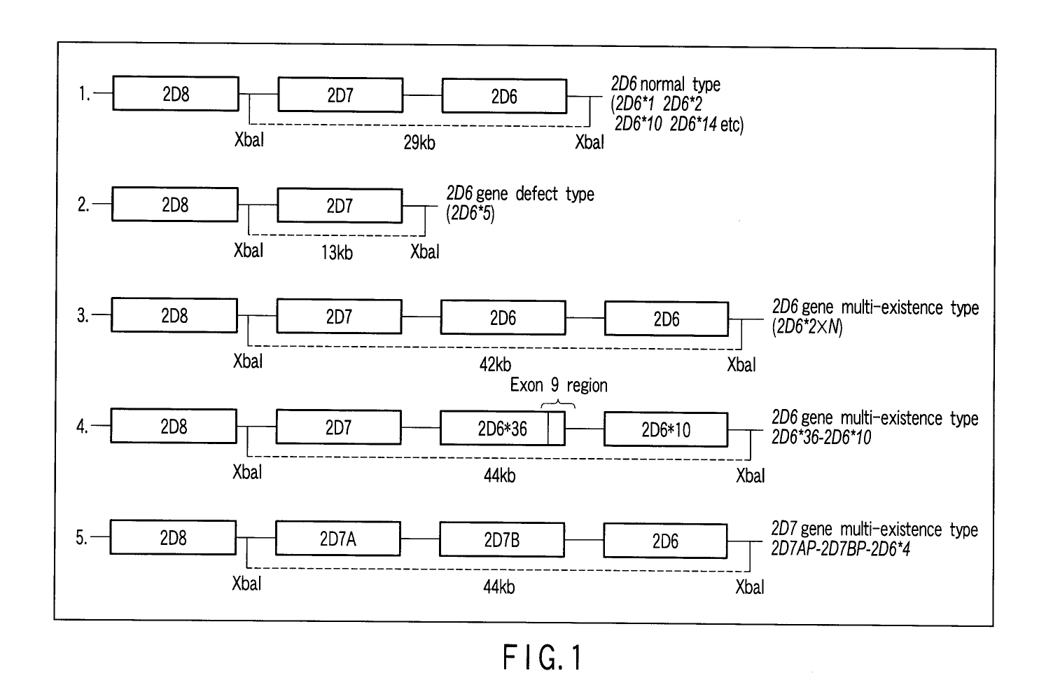

FIG. 1 is a schematic diagram of gene structures showing mutant alleles concerned with the gene number of CYP2D6. In FIG. 1, CYP2D7 and CYP2D8 are pseudogenes having very high homology to CYP2D6, as described above. The gene structure concerned with the gene number of CYP2D6 can be divided roughly into 5 types shown below. As used herein, the gene number is intended to mean the gene number of CYP2D6 having an enzyme activity. The gene number of every normal type is counted as 1, and the normal type includes alleles with no enzyme activity due to a mutation in the 2D6 gene. For example, even normal types may have no enzyme activity owing to mutations in the 2D6 gene, as is the case with 2D6*3, *4, *6, *7, *8, *11, *12, *14, *15, *18 and *21, for example. In the present invention, their gene number is also counted as 1. Mutations in the 2D6 gene should be separately examined by the existing method such as the Taq-man method, invader method, SSCP method, PCR-RFLP, allele specific primer PCR method or allele specific oligonucleotide hybridization method.

1. Normal type. The gene number of CYP2D6 is 1. A CYP2D6 gene defect or multi-existence is not observed. A 29-kbp band is obtained in Southern blot analysis with restriction enzyme XbaI (XbaI-RFLP (restriction fragment length polymorphism) analysis).

2. Defect type. The gene number of CYP2D6 is 0. This allele is designated CYP2D6*5. A 13- or 11.5-kbp band is obtained in XbaI-RFLP analysis. This type appears frequently in Orientals.

3. Multi-existence type. The gene number of CYP2D6 is 2 or more. This allele is designated CYP2D6*2×N. In XbaI-RFLP analysis, a 42-kbp band is obtained when N=2, a 54-kbp band when N=3, and a 175-kbp band when N=13.

4. Multi-existence type of CYP2D6*36-CYP2D6*10. The gene number is 1, because CYP2D6*36 does not have an enzyme activity. A 44-kbp band is obtained in XbaI-RFLP analysis. This allele appears frequently in Orientals.

5. CYP2D7 multi-existence type. The gene number is 1. This allele is designated CYP2D7-CYP2D7-CYP2D6. A 44-kbp band is obtained in XbaI-RFLP analysis (Heim et al., GENOMICS 14, 49-58, 1992).

CYP2D6*10 is an allele highly frequently appearing in Orientals. It is reported that this allele appears with a frequency of about 38% among the Japanese (Yuko Nishida et al., Pharmacogenetics, 10:567-570, 2000) and about 50% among the Chinese (Johansson et al., MOLECULAR PHARMACOLOGY, 46:452-459, 1994). At least 80% of alleles carrying this CYP2D6*10 carry CYP2D6*36 (Soyama et al., Drug Metab. Pharmacokinet. 21 (3): 208-216, 2006). That is, this allele belongs to the type described in 4 above.

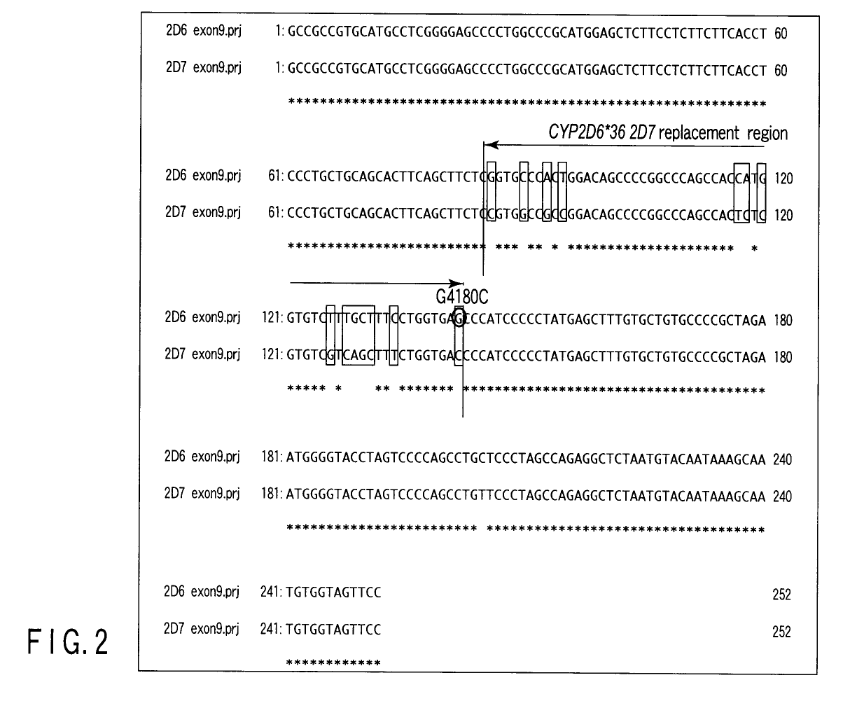

This CYP2D6*36 is a mutant allele wherein a part of Exon 9 region of the CYP2D6 gene is replaced by a sequence of the CYP2D7 gene. This CYP2D6*36 does not have an enzyme activity. FIG. 2 shows a part of the nucleotide sequence of Exon 9 region in each of the CYP2D6 gene and CYP2D7 gene. In CYP2D6*36, 13 bases in the region (CYP2D6*36 2D7 substitution region) indicated by arrows in FIG. 2 are replaced by the same bases as in the CYP2D7 gene (Johansson et al., 1994).

CYP2D6*5, *10, and *36 appear particularly highly frequently in Orientals, and thus the accurate determination and discrimination of their 1 to 5 mutant alleles are important. For accurate determination of the enzymatically active gene number, it is required that CYP2D6*36 be not counted.

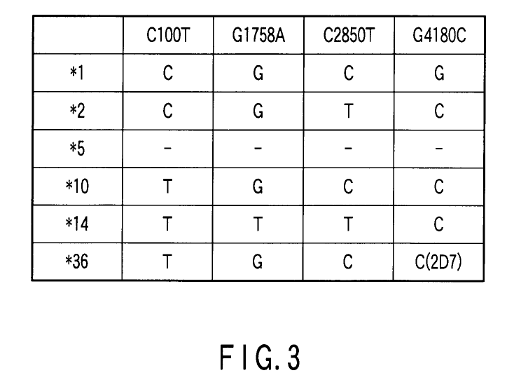

Other alleles appearing highly frequently among the Japanese are CYP2D6*1, *2, *5, *10, *14 and *36. FIG. 3 shows single nucleotide polymorphism in the CYP2D6 gene. As shown in FIG. 3, CYP2D6*1, *2, *10 and *14 can be detected by examining C100T, G1758A, C2850T and G4180C. However, CYP2D6*5 and *36 cannot be detected or discriminated even by examining single nucleotide polymorphism. “C(2D7)” in *36 in FIG. 3 is indicated to be the same as base 4180 in 2D7.

As the method of detecting a CYP2D6 gene defect and multi-existence, a method that involves amplifying the CYP2D6 gene and CYP2D8 gene with primers common between them, and then comparing their amplified products, has been disclosed as described above in the related art. In this method, however, the primers are designed toward Exon 6 region, the gene number of enzyme activity-free CYP2D6*36 is also inevitably counted as the gene number of CYP2D6.

Accordingly, the present inventors used a primer consisting of a sequence common between the CYP2D6 gene and CYP2D8 gene but different from the CYP2D7 gene and containing a part or all of bases at the 86-, 90- and 93-positions in Exon 9 region of the CYP2D6 gene, thereby succeeding in specifically amplifying the CYP2D6 gene and CYP2D8 gene only without amplifying CYP2D6*36.

In this specification, bases in the CYP2D6 gene are numbered starting from the translation initiation site. It is noted that the above-mentioned C100T, C2850T and G4180C are expressed as C188T, C2938T and G4268C respectively when shown by numbering from the translation initiation site.

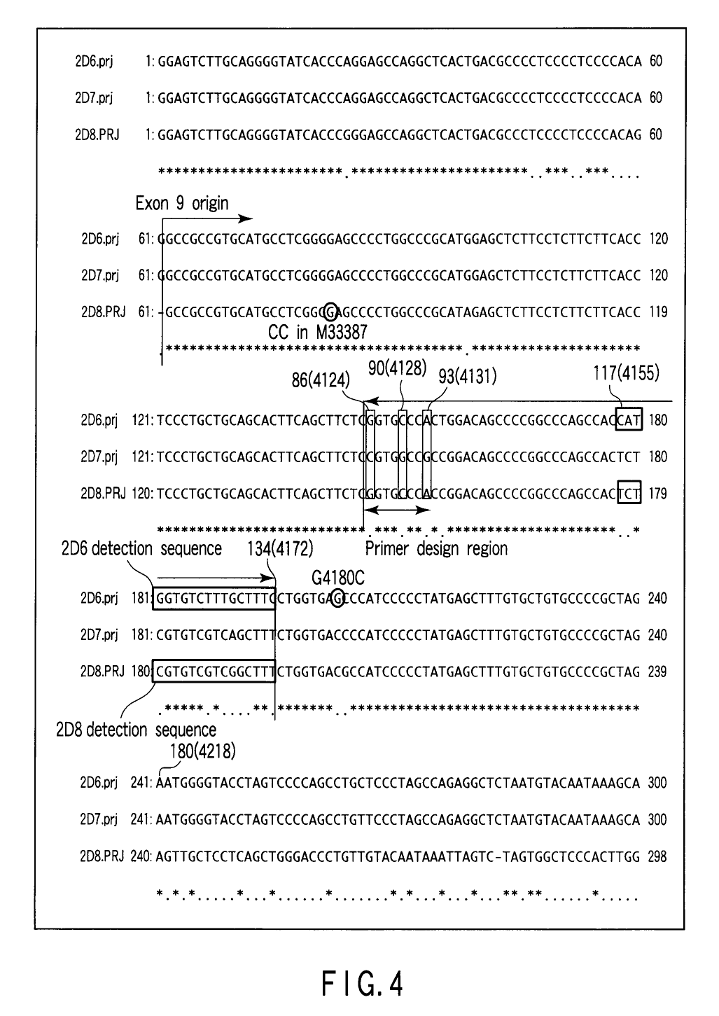

FIG. 4 shows nucleotide sequences around Exon 9 region in each of the CYP2D6, CYP2D7 and CYP2D8 genes. CYP2D6 consists of a sequence under Gene Bank Accession No. M33388. CYP2D7 consists of a sequence under Gene Bank Accession No. X58467. CYP2D8 is based on a sequence under Gene Bank Accession No. M333887. In the CYP2D8 sequence registered under Gene Bank Accession No. M333887, the portion in the second line indicated by circle was CC, but when 14 Japanese samples purchased from Coriell Cell Repositories were analyzed for sequence, that portion was G in all samples.

The bases in the 2D7 substitution region in CYP2D6*36, which are common between the CYP2D6 and CYP2D8 genes but are different from the CYP2D7 gene, are bases at the 86-, 90- and 93-positions from the initiation base of Exon 9 in the CYP2D6 gene (bases at the 4124-, 4128- and 4131-positions when counted from the translation initiation site of CYP2D6). It follows that when a primer containing a part or all of these 3 bases is used, the CYP2D6 gene and CYP2D8 gene only are specifically amplified. That is, the amplification of not only the CYP2D7 gene but also CYP2D6*36 can be prevented.

Another primer is not particularly limited, but is preferably a sequence complementary to a sequence common between the CYP2D6 gene and CYP2D8 gene. A region downstream of a base at the 180-position of Exon 9 region in the CYP2D6 gene is poor in the sequence homology of the CYP2D6 gene to the CYP2D8 gene. Accordingly, another primer is designed preferably toward a region upstream of a base at the 180-position.

When a sequence different between the CYP2D6 gene and CYP2D8 gene is used, mix bases or universal bases such as deoxyinosine (dl) may be used. This primer may also be complimentary to the CYP2D7 gene. As long as one of the pair of primers contains a part or all of the above 3 bases, the CYP2D6 gene and CYP2D8 gene only can be specifically amplified even if the other primer is common among the CYP2D6 gene, CYP2D8 gene and CYP2D7 gene.

Hereinafter, the procedures of the detection method of the invention are described. In this specification, a nucleic acid to be subjected to detection is referred to as target nucleic acid. A region to be amplified within the CYP2D6 gene is referred to as CYP2D6 target nucleic acid region, and its amplified product is referred to as CYP2D6 amplified product. A region to be amplified within the CYP2D8 gene is referred to as CYP2D8 target nucleic acid region, and its amplified product is referred to as CYP2D8 amplified product.

In the present invention, a nucleic acid obtained from a sample is amplified with a pair of the primers described above. By this amplification process, the CYP2D6 target nucleic acid region and the CYP2D8 target nucleic acid region are amplified to yield a CYP2D6 amplified product and a CYP2D8 amplified product, respectively. The amplification can be carried out by any method known in the art. Examples of the method that can be used in the invention include polymerase chain reaction (PCR), Nucleic Acid Sequence-Based Amplification (NASBA), Strand Displacement Amplification (SDA), Rolling Circle Amplification (RCA), Ligase chain reaction (LCR), Isothermal and Chimeric primer-initiated Amplification of Nucleic Acids (ICAN) and Loop-mediated isothermal amplification (LAMP).

Then, the amounts of the CYP2D6 amplified product and CYP2D8 amplified product are determined. This determination can be carried out by any known method. For example, the Taq-man method, the pyrosequence method, the invader method or a determination method using DNA chips is used.

In determination of the amounts of the amplified products, detection sequences specific for the CYP2D6 amplified product and CYP2D8 amplified product respectively are preferably detected. In this specification, the sequences used for detection of the respective amplified products are referred to as CYP2D6 detection sequence and CYP2D8 detection sequence, respectively.

FIG. 4 shows one embodiment of regions used in the detection sequences. The detection sequence may be a part or the whole of the region shown in FIG. 4, but is not limited thereto. For example, a sequence of from a base at the 117-position to a base at the 134-position counted from the initiation base of Exon 9 (that is, between a base at the 4155-position and a base at the 4172-position counted from the translation initiation site of CYP2D6) is used preferably as the CYP2D6 detection sequence. For example, a sequence of from a base at the 117-position to a base at the 134-position counted from the initiation base of Exon 9 is used preferably as the CYP2D8 detection sequence.

When the detection sequence is a part or all of the sequence of from a base at the 117-position to a base at the 134-position from the initiation base of Exon 9, one primer should be designed as a reverse primer toward a region of from the initiation base of Exon 9 to the 135-position or thereafter. This reverse primer is designed preferably toward a region of from the 135-position to the 180-position.

In the Taq-man method, probes complementary to the respective detection sequences, that is, the CYP2D6 detection probe and CYP2D8 detection probe, are used to determine the amounts of the respective amplified products. In the pyrosequence method, a common sequence primer binding to a region upstream of each detection sequence is used. The amounts of the respective amplified products can be determined by adding bases unique to each of CYP2D6 and CYP2D8 and then determining the reaction amounts.

Alternatively, the amplified product can be detected by hybridization with a probe containing a sequence complementary to the respective detection sequences. This probe is preferably immobilized on a substrate before use. For example, a DNA chip or a DNA microarray is preferably used as a substrate for immobilizing the probe.

Then, the amount of the amplified product of the CYP2D6 gene is compared with the amount of the amplified product of the CYP2D8 gene. As described above, the gene number of CYP2D8 is 2. Accordingly, the gene number of CYP2D6 can be determined by comparing the amount of the amplified product of the CYP2D6 gene with that of the CYP2D8 gene.

As described above, the primers defined in the present invention can be used to determine the accurate gene number of CYP2D6 without counting CYP2D6*36. A defect and multi-existence mutation in the CYP2D6 gene can thereby be detected very easily in a short time.

Thereafter, the mutation in the CYP2D6 gene is separately examined by the existing method such as the Taq-man method, the invader method, the SSCP method, the PCR-RFLP method, the allele specific primer PCR method, or the allele specific oligonucleotide hybridization analysis, whereby the genotype can be analyzed in more detail.

### EMBODIMENTS

Hereinafter, the determination of the amplified products by using a nucleic acid-immobilized substrate is described. The nucleic acid probe contains a sequence complementary to the detection sequence, as described above. The nucleic acid probe is made of, but is not limited to, DNA, RNA, PNA, LNA, a nucleic acid having a methyl phosphonate skeleton, and other artificial nucleic acids. For immobilization on a substrate, the terminus of the nucleic acid probe may be modified with a reactive functional group such as an amino group, a carboxyl group, a hydroxyl group, a thiol group or a sulfone group. A spacer may be introduced into between the functional group and the nucleotide. For example, a spacer consisting of an alkane or ethylene glycol skeleton may be used.

<Nucleic Acid Probe-Immobilized Substrate>

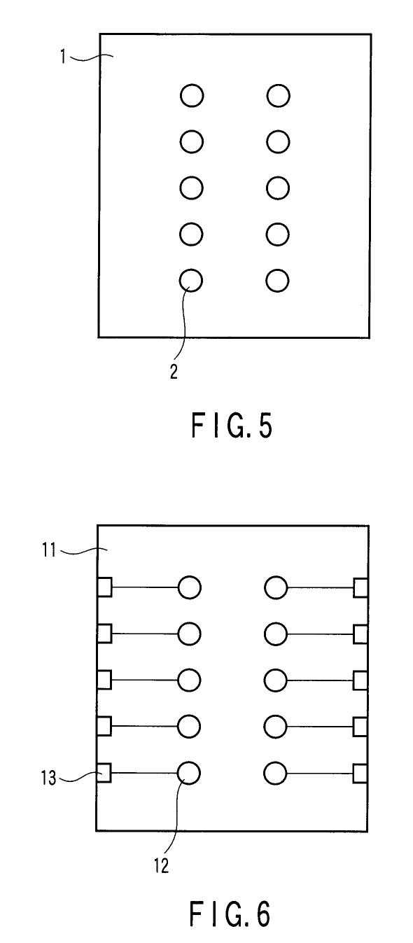

A schematic diagram of the nucleic acid probe-immobilized substrate in one embodiment is shown in FIG. 5. The nucleic acid probe is immobilized in an immobilization region 2 on a substrate 1. The substrate 1 can be produced for example from a silicon substrate or the like, but the material of the substrate is not limited thereto. The nucleic acid probe may be immobilized by a means known in the art. One kind of nucleic acid probe or a plurality kind of nucleic acid probes may be immobilized on one substrate 1, and the arrangement and number of the kinds may be suitably designed and changed as necessary by those skilled in the art. When the nucleic acid probe is fluorescently detected as described later, the nucleic acid probe-immobilized substrate such as in this embodiment is preferably used.

FIG. 6 shows a schematic diagram of the nucleic acid-immobilized substrate in another embodiment. In the embodiment, a substrate 11 is equipped with an electrode 12. The nucleic acid probe is immobilized on the electrode 12. The electrode 12 is connected to a pad 13 for transmitting electrical information. The substrate 11 can be produced for example from a silicon substrate or the like, but the material of the substrate is not limited thereto. Production of the electrode and immobilization of the nucleic acid probe may be conducted by a means known in the art. The electrode may be produced from, but are without limited to, a single metal or an alloy thereof such as gold, a gold alloy, silver, platinum, mercury, nickel, palladium, silicon, germanium, gallium or tungsten, carbon such as graphite or glassy carbon, or an oxide or compound thereof.

The immobilized substrate in FIG. 6 has 10 electrodes, but the number of electrodes arranged on one substrate is not limited and may be arbitrarily changed. The pattern of electrodes arranged thereon is not limited to that shown in FIG. 6 and may be suitably designed and changed as needed by those skilled in the art. The substrate 11 may be equipped with a reference electrode and a counter electrode if necessary. When the probe is electrochemically detected as described later, the probe-immobilized substrate such as in this embodiment is preferably used.

<Hybridization Between the Nucleic Acid Probe and the Amplified Product>

Hybridization between the nucleic acid probe and the amplified product is conducted under suitable conditions. Suitable conditions vary depending on the type and structure of the amplified product, the type of bases contained in the detection sequence, and the type of nucleic acid probe. Hybridization is conducted for example in a buffer solution with an ionic strength in the range of 0.01 to 5 and in the range of pH 5 to 10. Dextran sulfate that is a hybridization accelerator, salmon sperm DNA, calf thymus DNA, EDTA and a surfactant may be added to the reaction solution. The reaction is carried out for example at a temperature in the range of 10 to 90° C., and the efficiency of the reaction may be increased with stirring or shaking. For washing after the reaction, a buffer solution with an ionic strength in the range of 0.01 to 5 and in the range of pH 5 to 10, for example, may be used.

<Detection>

When the nucleic acid probe immobilized on the substrate is hybridized with the amplified product, a double-stranded nucleic acid is formed. This double-stranded nucleic acid can be electrically or fluorescently detected.

(a) Electric Current Detection Method

A method of electrochemically detecting a double-stranded nucleic acid is described. In this method, a double-stranded chain-recognizing substance that specifically recognizes a double-stranded nucleic acid is used. Examples of the double-stranded chain-recognizing substance include, but are not limited to, Hoechst 33258, acridine orange, quinacrine, daumonycin, metallointercalator, bisintercalator such as bisacridine, trisintercalator, and polyintercalator. These substances may further be modified with an electrochemically active metal complex such as ferrocene or viologen.

The concentration of the double-stranded chain-recognizing substance suitable for use varies depending on its type, but is generally in the range of 1 ng/mL to 1 mg/mL. In this case, a buffer solution with an ionic strength of 0.001 to 5 and in the range of pH 5 to 10 may be used.

During or after the hybridization reaction, the double-stranded chain-recognizing substance is added to the reaction solution. When a double-stranded nucleic acid has been formed by hybridization, the double-stranded chain-recognizing substance binds thereto. It follows that by applying a voltage greater than or equal to the voltage causing an electrochemical reaction of the double-stranded chain-recognizing substance, a reaction current derived from the double-stranded chain-recognizing substance can be determined. In this case, constant-rate voltage, pulsed voltage or constant voltage may be applied. In determination, the current and voltage may be regulated by using apparatuses such as a potentiostat, a digital multi-meter and a function generator. For example, a known electrochemical detection means described in JP-A 1998-146183 (KOKAI) can be preferably used.

(b) Fluorescence detection method

A method of fluorescently detecting a double-stranded nucleic acid is described. A primer is previously labeled with a fluorescently active substance. Alternatively, a secondary probe labeled with a fluorescently active substance is used in detection. A plurality of labels may be used. The fluorescently active substance includes, but is not limited to, fluorescent dyes such as FITC, Cy3, Cy5 and rhodamine. The fluorescent substance is detected for example with a fluorescence detector. An appropriate detector adapted to the type of label is used to detect the labeled detection sequence or secondary probe.

<Determination by the LAMP Method>

In one embodiment of the present invention, the LAMP method is used. The LAMP method is a technique of amplifying a nucleic acid under an isothermal condition at 60 to 65° C. The LAMP method is advantageous over the PCR method in that an amplified product can be obtained more rapidly in a larger amount.

In the LAMP method, 4 kinds of primers recognizing 6 regions of the target nucleic acid, a chain-substitution-type DNA synthetase, and a substrate are used. A loop structure is formed in an amplified product by the LAMP method. Further, repetitive sequences of various sizes are formed on the same chain. The sequences of the repetitive sequences are complementary to each other.

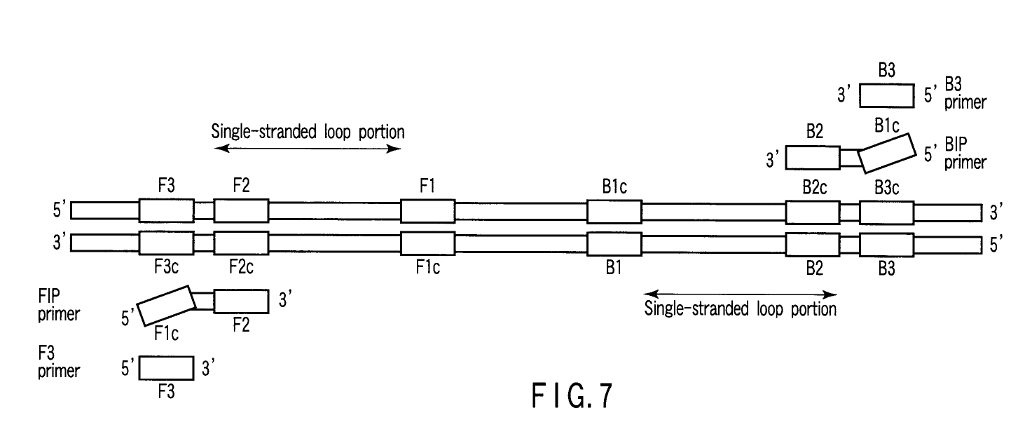

Hereinafter, the LAMP method is outlined. In the LAMP method, an F3 region, F2 region and F1 region are set out in this order from the 5′-terminal side of the target nucleic acid, and a B3c region, B2c region and B1c region are set out in this order from the 3′-terminal side. Four kinds of primers as shown in FIG. 7 are used to amplify the target nucleic acid. F1c, F2c, F3c, B1, B2 and B3 regions refer respectively to regions, in a complementary chain, of F1, F2, F3, B1c, B2c and B3c regions.

The 4 kinds of primers used in amplification of the nucleic acid in the LAMP method are (1) FIP primer having, at its 3′-terminal side, the same sequence as the F2 region and having, at its 5′-terminal side, a complementary sequence to the F1 region; (2) F3 primer consisting of the same sequence as the F3 region; (3) BIP primer having, at its 3′-terminal side, a complementary sequence to the B2c region and having, at its 5′-terminal side, the same sequence as the B1c region; and (4) B3 primer consisting of a complementary sequence to the B3c region. Generally, the FIP primer and BIP primer are called inner primers, and the F3 primer and B3 primer are called outer primers.

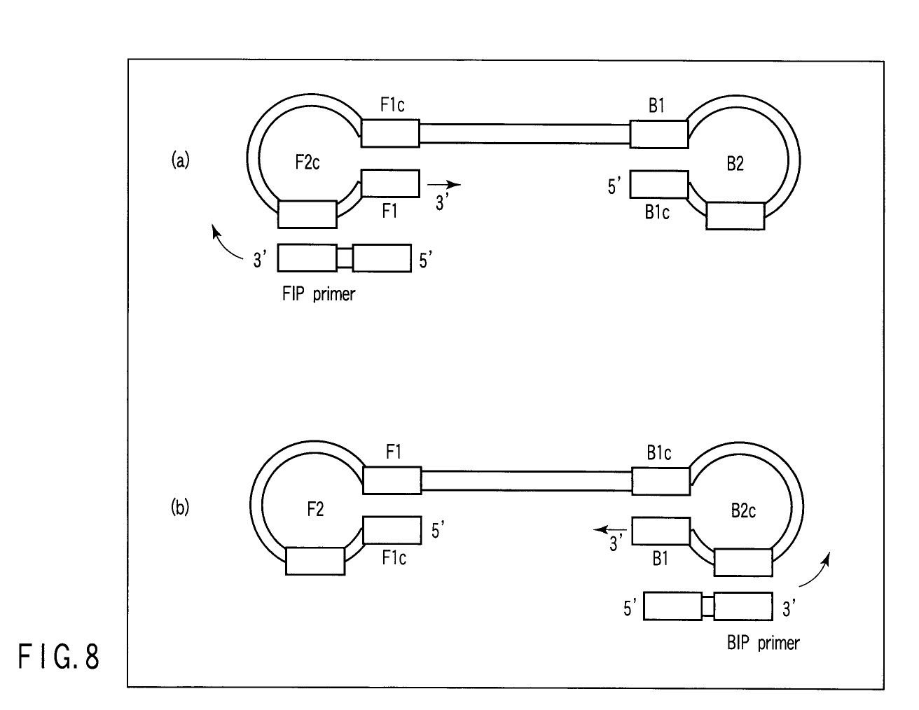

When the 4 kinds of primers are used in LAMP amplification, an intermediate product having a dumbbell structure as shown in FIG. 8 is formed. The FIP and BIP primers bind to F2c and B2c regions in the single-stranded loop, to initiate an elongation reaction from the 3′-terminus of the primer and from the 3′-terminus of the intermediate product. For details, refer to Japanese Patent No. 3313358.

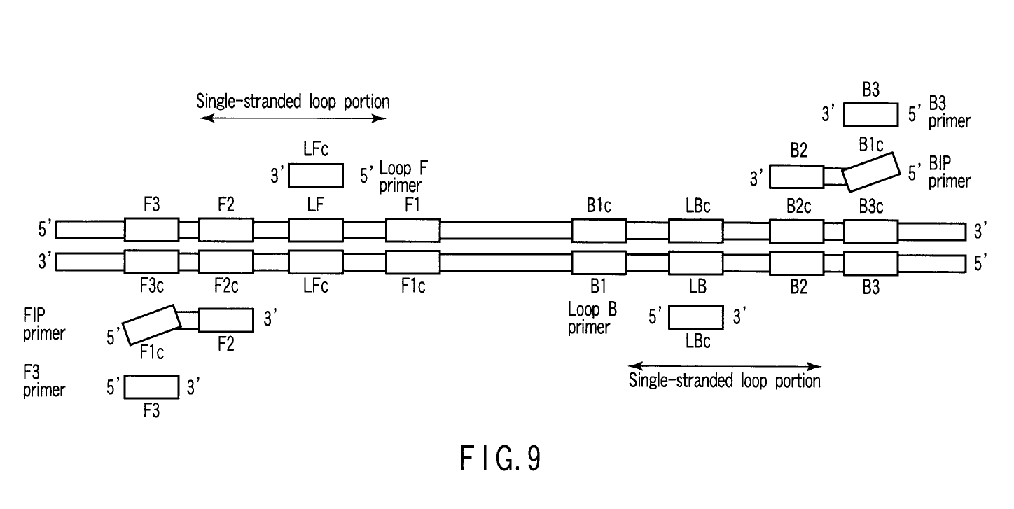

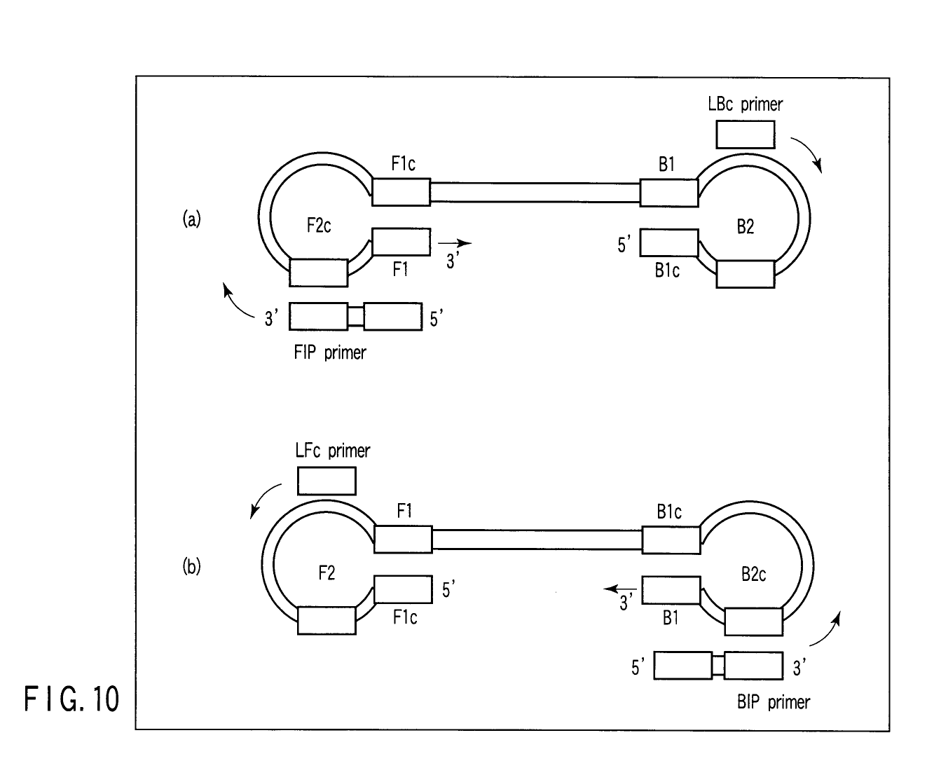

In the LAMP method, a primer called a loop primer can further be arbitrarily used to shorten the amplification time. In this case, as shown in FIG. 9, an LF region is set out in a portion ranging from the F2 region to F1 region, and an LBc region is set out in a portion ranging from the B2c region to B1c region. These portions are referred to as loop primer regions. Then, a loop primer LFc consisting of a complementary sequence to the LF region, and a loop primer LBc consisting of the same sequence as the LBc region are used in addition to the 4 kinds of primers described above. For details, reference is made to WO2002/024902. The loop primers LFc and LBc may be simultaneously used, or only one of them may be used. The loop primer anneals a loop different from a loop annealed by the FIP or BIP primer, as shown in FIG. 10, to provide a further synthesis origin thereby promoting amplification.

<Detection of LAMP Amplified Product>

In the LAMP amplified product, there is a single-stranded region. In FIG. 7, a single-stranded chain is formed between the F2 and F1 regions (including the F2 region) and between the B2 and B1 regions (including the B2 region). This single-stranded portion can be conveniently used for hybridization with the probe (JP-A 2005-143492(KOKAI)). Accordingly, each primer is designed such that the detection sequence detected by the probe is sited on this single-stranded portion.

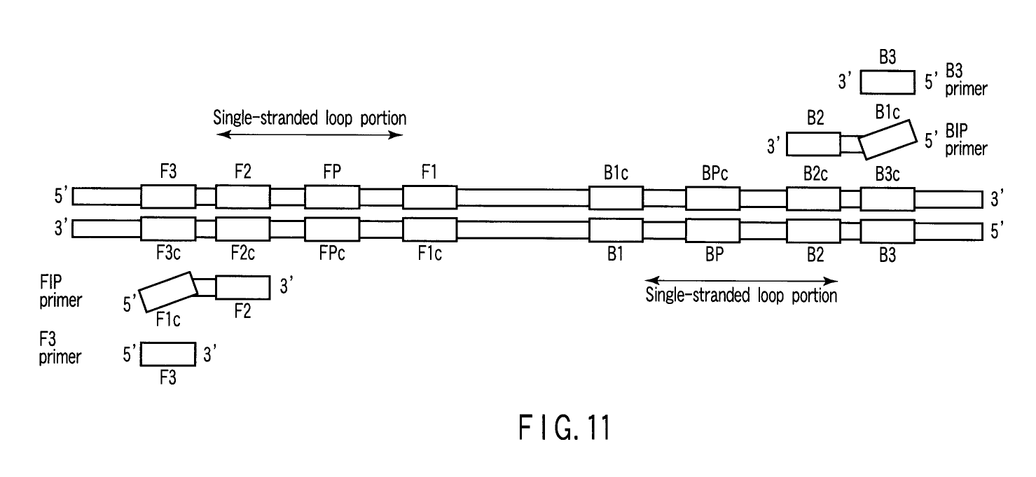

As shown in FIG. 11, the detection sequence FP region is set out between the F2 region and F1 region, and similarly the detection sequence BP region is set out between the B2 region and B1 region. The FPc region and BPc region are complementary chains to the FP region and BP region, respectively, and one or more of the FP region, BP region, FPc region and BPc region can be used as the detection sequences binding to the probe. However, there is the LF region besides the FP region between the F2 region and F1 region, and similarly there is the LB region besides the BP region between the B2 region and B1 region, and therefore, the detection sequences and loop primers should be designed such that there is no overlap between the FP region and LF region or between the BP region and LB region.

<Establishment of Regions for the Lamp Method>

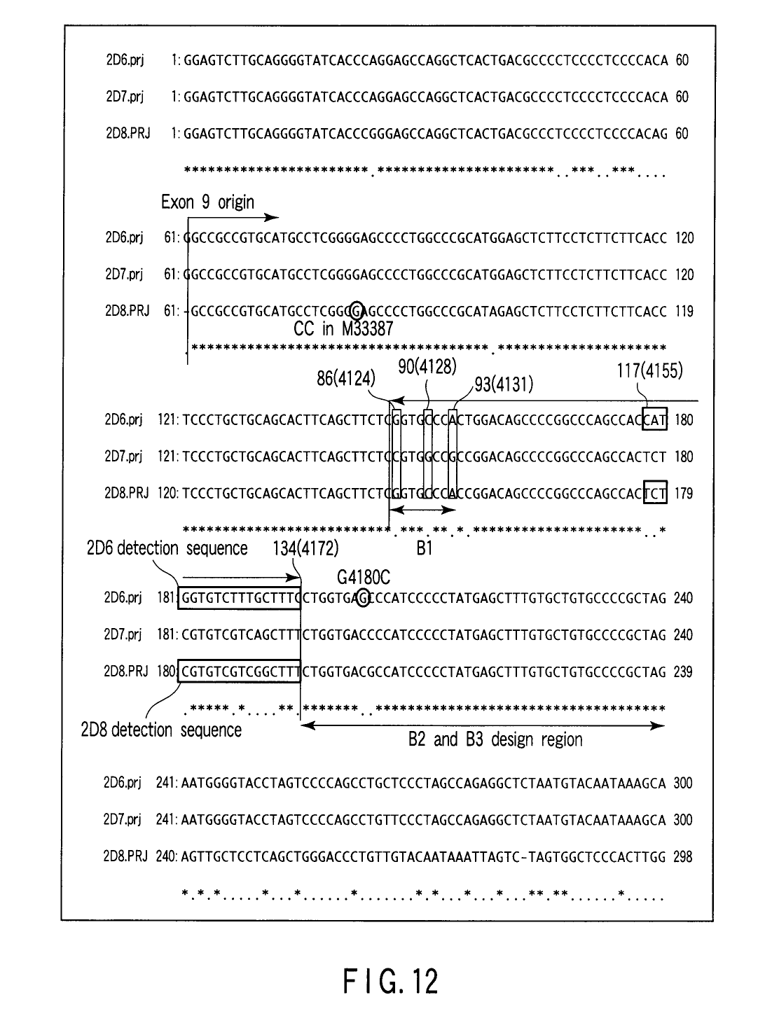

As shown in FIG. 12, the homology between CYP2D6 and CYP2D8 is decreased in sequences downstream of the 181-position from the initiation base of Exon 9 in CYP2D6. Accordingly, six LAMP primer design regions for F1, F2, F3, B1c, B2c and B3c are designed preferably upstream of a base at the 180-position. Further, the primer is designed to contain one or more position of the 86-, 90- and 93-positions from the initiation base of Exon 9 in CYP2D6. Further, the detection sequences of CYP2D6 and CYP2D8 are designed so as to contain a part or the whole of a region of from the 117- to 134-positions from the initiation base of their Exon 9. Because there is such limitation, a region containing one or more position of the 86-, 90-, and 93-positions from the initiation base of Exon 9 in CYP2D6 is made B1c region, and the detection sequence is designed so as to be sited between B1c and B2c. The detection sequence is designed between B1c and B2c, and thus the loop primer is designed preferably between the F2 region and F1 region.

In a preferable mode of the invention, the gene is amplified by the LAMP method, and the amplified product is determined by using a current-detection-type DNA chip. In this mode, a defect or multi-existence of the CYP2D6 gene can be detected particularly rapidly and easily.

<Improvement in Detection Accuracy by Adding a Block Nucleic Acid>

When the gene number of CYP2D6 is 1, 2, 3, and more than 3, the CYP2D8 amplification amount: CYP2D6 amplification amount ratio is 2:1, 2:2, 2:3, and 2: more than 3, respectively. When the number of the detection sequences in the LAMP product is too large relative to the number of the detection probes, almost all probes bind to the LAMP product. Accordingly, a signal obtained from the probes is saturated, and as a result, there may be no difference between the obtained signal of CYP2D8 and the obtained signal of CYP2D6.

Accordingly, it is desirable that a nucleic acid containing a sequence complementary to a part or all of the CYP2D6 detection sequence and CYP2D8 detection sequence is added to the hybridization solution. The nucleic acid is referred to as block nucleic acid. The block nucleic acid added to the reaction solution binds to the detection sequence in the LAMP product. Thus the detection sequence become double-stranded, and so hardly hybridizes with the probe. Hence, the number of detection sequences that bind to the probe can be regulated by adding the block nucleic acid.

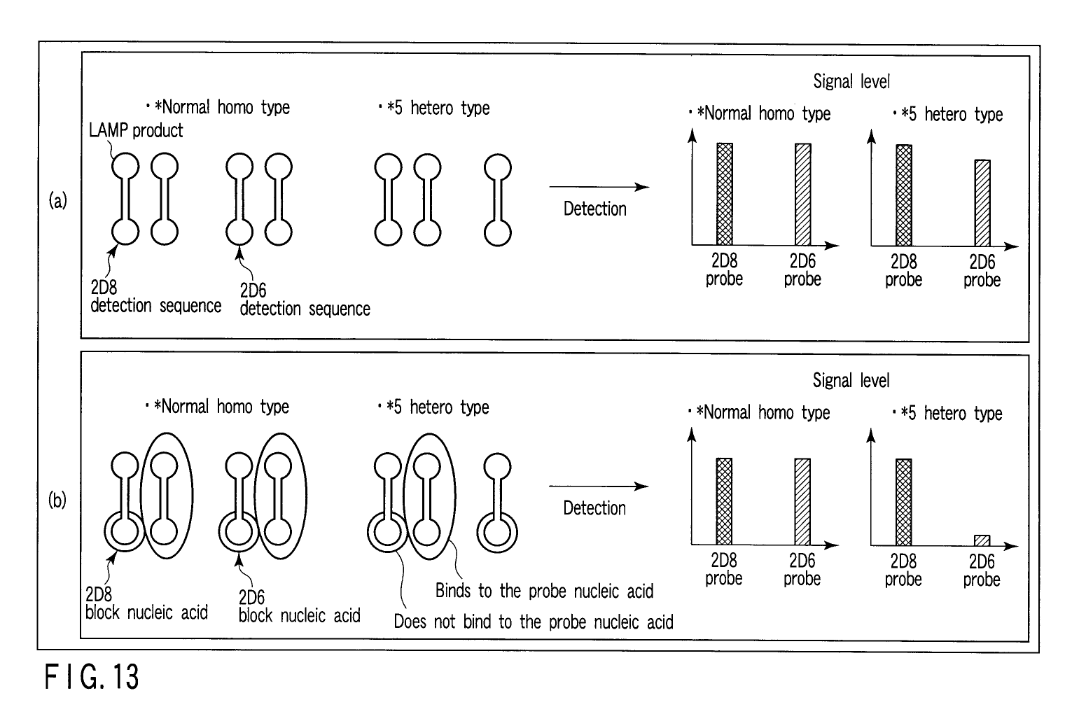

FIG. 13 shows a conceptual diagram of the block nucleic acid. The CYP2D8 amplification amount: CYP2D6 amplification amount ratio in a sample of CYP2D6 whose gene number is 2 (normal homo type) is 2:2. The ratio in a sample of CYP2D6 whose gene number is 1 (*5 hetero type) is 2:1. When the LAMP product is hybridized with the probes without being processed, the amplified product binds to almost all of the probes if the number of the detection sequences in the LAMP product is too large relative to the number of the probes, as shown in FIG. 13A. Therefore, there is no difference between the signal pattern of *5 hetero type and the signal pattern of normal homo type. Accordingly, the case where the amplification amount ratio is 2:2 and the case where the amplification amount ratio is 2:1 cannot be clearly distinguished from each other.

FIG. 13B shows the case where CYP2D6 block nucleic acids, the number of which is almost the same as the number of the CYP2D6 detection sequences, were added, and CYP2D8 block nucleic acids, the number of which is almost the same as the number of the CYP2D8 detection sequences, were added. In this case, the amplification amount ratios of 2:2 and 2:1 become 1:1 and 1:0 respectively. Accordingly, the signal of CYP2D6 is hardly generated in *5 hetero type, so a significant difference from the signal pattern of normal homo type can be recognized. The case where the amplification amount ratio is 2:2 can be clearly distinguished from the case where the amplification amount ratio is 2:1.

The amount of the block nucleic acid for CYP2D6 and the amount of the block nucleic acid for CYP2D8 can be suitably changed. Both the block nucleic acids may not be added in equal amounts and may be added at different concentrations. The block nucleic acid includes, but is not limited to, DNA, RNA, PNA, LNA, a nucleic acid having a methyl phosphonate skeleton, and other artificial nucleic acids.

<Test Sample>

The sample intended in the present invention is not particularly limited; for example, materials collected from humans, such as blood, serum, leucocytes, hair roots and oral mucosa can be used. From these test samples, nucleic acid components are extracted to give the target nucleic acid subjected to the detection test. A solution containing the target nucleic acid including the CYP2D6 gene, CYP2D8 gene etc. is referred to as a sample solution. The extraction method is not particularly limited, but a commercial nucleic acid extraction tool QIAamp (manufactured by QIAGEN), Smitest (manufactured by Sumitomo Metal Industries, Ltd.) and the like may also be used.

According to another aspect of the invention, there is provided a kit having a pair of the above primers for use in the detection method of the present invention. There is also provided a kit having primers for the LAMP method. The kit may include a chain-substitution-type DNA synthetase, a synthetic substrate and a buffer solution. The kit may further include a probe complementary to the detection sequence.

There is also provided a probe-immobilized substrate, on which a probe complementary to the detection sequence is immobilized, for use in the detection method of the present invention. The probe-immobilized substrate is provided preferably as a DNA chip or a DNA microarray.

### EXAMPLES

### Comparative Example

### Genotype Analysis of 19 Samples of Japanese Genome

According to conventional methods, the CYP2D6 genotype of 19 samples of Japanese genome was determined. Southern blot analysis, PCR-RFLP analysis and nested PCR analysis were conducted.

(A) Southern Blot Analysis

The DNA (3 μg) was treated with XbaI and then electrophoresed on 0.5% agarose gel. Thereafter, the result of electrophoresis was transferred onto a nylon membrane (manufactured by Boehringer). A DIG-labeled CYP2D6 cDNA probe was used in hybridization, and detection was performed according to a standard protocol of DIG System (provided by Boehringer).

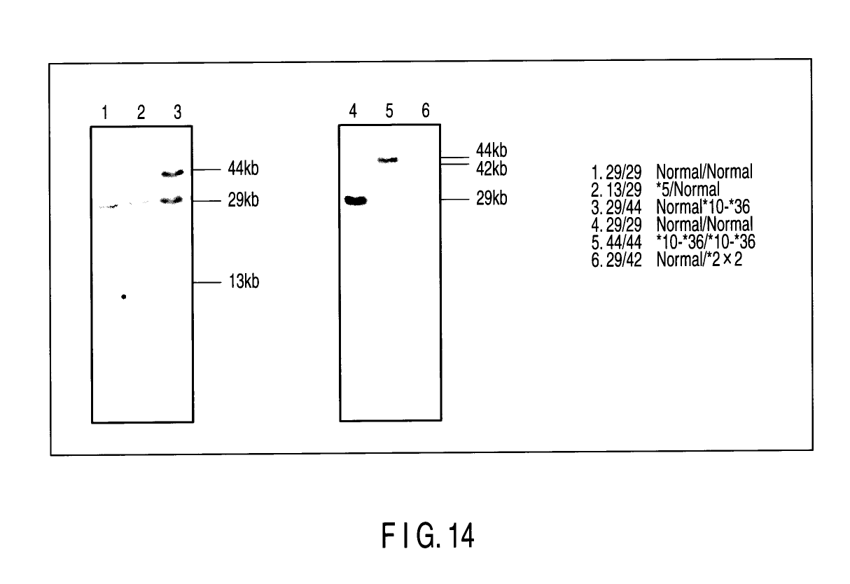

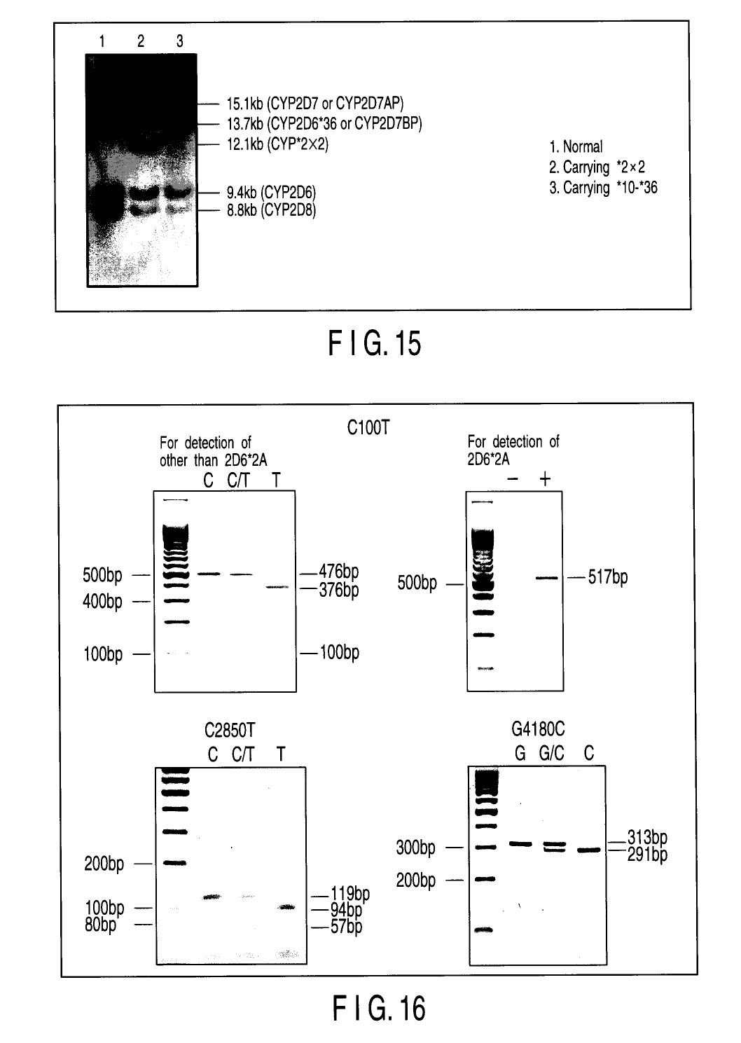

The results are shown in FIG. 14 (XbaI-RFLP analysis). Band sizes of 13 kbp, 29 kbp, 42 kbp and 44 kbp were confirmed. From this result, the presence of genotypes 2D6*5, normal type, 2D6*2×2, and 2D6*36-*10 was determined. However, bands of 42 kbp and 44 kbp are located adjacent to each other and can thus not be clearly distinguished from each other. Accordingly, EcoRI-RFLP analysis was further conducted. The DNA (3 μg) was treated with EcoRI and then electrophoresed on 0.8% agarose gel. The result is shown in FIG. 15. Bands of 12.1 kbp and 13.7 kbp were confirmed. From this result, 2D6*2×2 and 2D6*36-*10 can be clearly distinguished from each other.

(B) PCR-RFLP Analysis

For detecting *1, *2, *10 and *14 appearing highly frequently among the Japanese, single nucleotide polymorphism C100T, C2850T and G4180C were analyzed by PCR-RFLP. Primers for each single nucleotide polymorphism are as follows:

C100T:
For detection of other than 2D6*2A
F primer: 5′-ACCAGGCCCCTCCACCGG-3′
R primer: 5′-TCTGGTAGGGGAGCCTCAGC-3′
For detection of 2D6*2A
F primer: 5′-ACCAGGCCCCTCCACCGG-3′
R primer: 5′-GTGGTGGGGCATCCTCAGG-3′

(Johansson et al., 1994, primer 9, primer 10, primer 10B)

C2850T:
F primer: 5′-GCAGCTTCAATGATGAGAACCTG-3′
R primer: 5′-GGGTGTCCCAGCAAAGTTCAT-3′
G4180C:
F primer: 5′-CCATGGTGTCTTTGCTTTCC-3′
R primer: 5′-AGAGTTGGGTCAGTGGGGGACATG-3′

The DNA was amplified with pyrobest DNA polymerase (TAKARA Bio) and its attached buffer under the conditions shown in Table 1. 30 ng of genome was added to 50 μl reaction solution.

TABLE 1
PCR primers and amplification conditions for PCR-RFLP analysis

| | | | | Primer | dNTP |
|---|---|---|---|---|---|
| Target | Primer | | Sequence | concentration | concentration |
| C100T | F | For detection | ACCAGGCCCCTCCACCGG | 40 pmol | 0.3 mM |
| | R | of other than | TCTGGTAGGGGAGCCTCAGC | 40 pmol | |
| | | 2D6*2A | | | |
| | F | For detection | ACCAGGCCCCTCCACCGG | 20 pmol | 0.4 mM |
| | R | of 2D6*2A | GTGGTGGGGCATCCTCAGG | 20 pmol | |
| C2850T | F | — | GCAGCTTCAATGATGAGAACCTG | 20 pmol | 0.4 mM |
| | R | | GGGTGTCCCAGCAAAGTTCAT | 20 pmol | |
| G4180C | F | — | CCATGGTGTCTTTGCTTTCC | 40 pmol | 0.4 mM |
| | R | | AGAGTTGGGTCAGTGGGGGACATG | 40 pmol | |

| | | | Enzyme | | Annealing | Extension | |
|---|---|---|---|---|---|---|---|
| Target | Primer | | (pyrobest) | Step | temperature (X) | time (Y) | Reaction amount |
| C100T | F | For detection | 1.25 U/50 μL | 2Step | 70° C. | 40 seconds | 50 μL |
| | R | of other than | | | | | |
| | | 2D6*2A | | | | | |
| | F | For detection | | 3Step | 66° C. | 40 seconds | |
| | R | of 2D6*2A | | | | | |
| C2850T | F | — | | 3Step | 60° C. | 30 seconds | |
| | R | | | | | | |
| G4180C | F | — | | 3Step | 58° C. | 30 seconds | |
| | R | | | | | | |

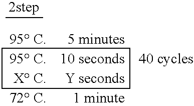

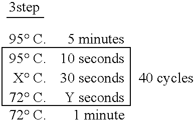

The resulting PCR products of C100T (for detection of other than 2D6*2A), C2850T and G4180C were cleaved with HphI, HpyCH4V and BstEII respectively. The observed band sizes of the respective types cleaved with the respective restriction enzymes are shown in Table 2. Whether 2D6*2A is present or not was judged by examining whether a 570-bp band appears or not as a result of amplification with the primer for detection of 2D6*2A.

TABLE 2
Restriction enzymes used for PCR-RFLP analysis and band pattern after
enzyme treatment

| SNP | C100T | | C2850T | | G4180C | |
|---|---|---|---|---|---|---|
| | (other than 2D6*2A) | | | | | |
| enzyme | HphI | | HpyCH4V | | BstEII | |
| Length | 517 bp | | 203 bp | | 313 bp | |
| before | | | | | | |
| treatment | | | | | | |
| Type | C | T | C | T | G | C |
| Length | 476 bp | 376 bp | 119 bp | 94 bp | 313 bp | 291 bp |
| after | 41 bp | 100 bp | 57 bp | 57 bp | | 22 bp |
| treatment | | 41 bp | 27 bp | 27 bp | | |
| | | | | 25 bp | | |

The results are shown in FIG. 16. As the marker, a 100-bp ladder (SIGMA Genosys) was used. Each single nucleotide polymorphism was clearly detected.

(C) Nested PCR Analysis

When a 44-kbp band is obtained in XbaI-RFLP analysis, the genotype is almost always 2D6*36-*10 in the case of the Japanese (Soyama et al., 2006). In Caucasians, however, it has reported that the genotype in the case is sometimes multi-existence-type of CYP2D such as CYP2D7AP-CYP2D7BP-CYP2D6.

To confirm the allele of 2D6*36-*10, nested PCR was carried out according to a method disclosed by Soyama et al. (2006). 2D6-7S and 2D62AS were used as 1st primers. 2D6E×7F6s and cyp32 were used as 2nd primers. The reaction conditions were the same as described in the literature.

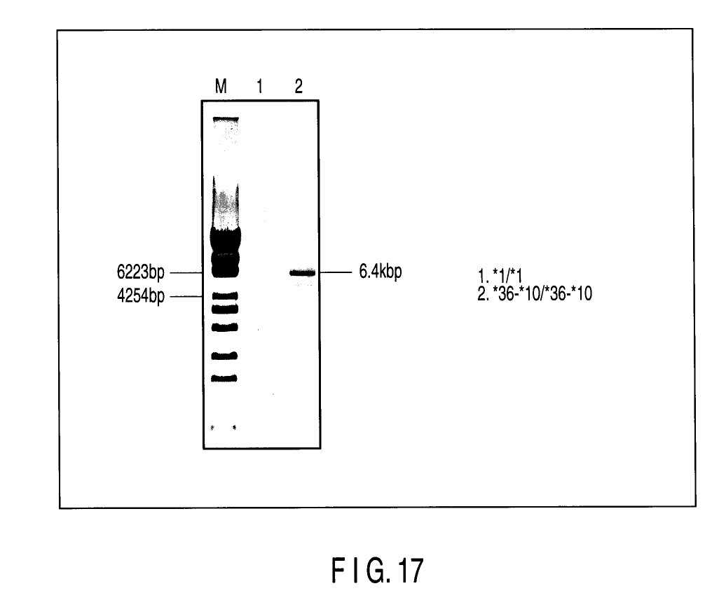

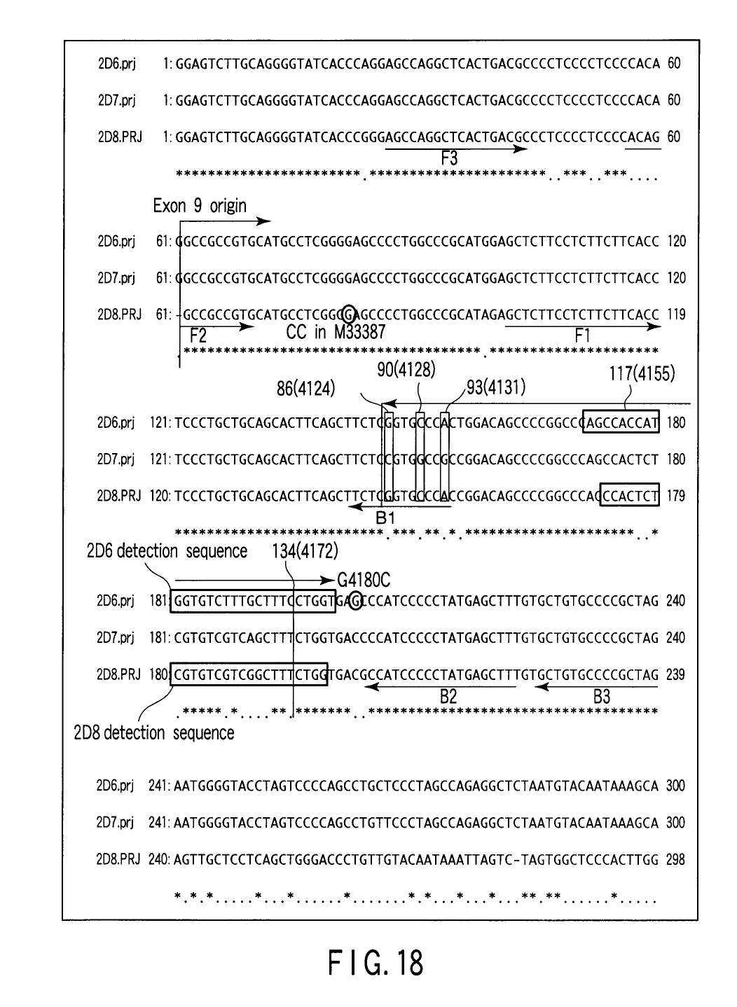

The results are shown in FIG. 17. λ-EcoT14 I digest (TAKARA Bio) was used as the marker. A 6.4-kbp band unique to 2D6*36-*10 was confirmed. The presence of 2D6*36-*10 could thereby be more clearly confirmed.

The genotypes of 19 Japanese samples determined by the comparative experiment described above are shown in Table 3.

TABLE 3
2D6 genotype of 19 Japanese samples

| | | Gene | | |
|---|---|---|---|---|
| Sample | | number | Number | |
| No. | genotype | of 2D6 | of *36 | XbaI-RFLP |
| 1 | *5/*5 | 0 | 0 | 13/13 |
| 2 | *1/*5 | 1 | 0 | 13/29 |
| 3 | *1/*5 | 1 | 0 | 13/29 |
| 4 | *5/*36-*10 | 1 | 1 | 13/44 |
| 5 | *5/*36-*10 | 1 | 1 | 13/44 |
| 6 | *1/*1 | 2 | 0 | 29/29 |
| 7 | *1/*1 | 2 | 0 | 29/29 |
| 8 | *1/*1 | 2 | 0 | 29/29 |
| 9 | *1/*10 | 2 | 0 | 29/29 |
| 10 | *2/*10 | 2 | 0 | 29/29 |
| 11 | *1/*36-*10 | 2 | 1 | 29/44 |
| 12 | *1/*36-*10 | 2 | 1 | 29/44 |
| 13 | *1/*36-*10 | 2 | 1 | 29/44 |
| 14 | *1/*36-*10 | 2 | 1 | 29/44 |
| 15 | *1/*36-*10 | 2 | 1 | 29/44 |
| 16 | *2/*36-*10 | 2 | 1 | 29/44 |
| 17 | *36-*10/*36-*10 | 2 | 2 | 44/44 |
| 18 | *36-*10/*36-*10 | 2 | 2 | 44/44 |
| 19 | *1/*2X2 | 2 | 2 | 29/42 |

A gene defect or multi-existence can be judged by Southern blot analysis. However, Southern blot analysis is very complicated in operation and requires a time as long as about 3 days for analysis. Accordingly, judgment of a gene defect or multi-existence by the conventional method is very difficult.

### Example

### Type Analysis of 19 Samples of Japanese Genome

According to the method of the present invention, a CYP2D6 gene defect and multi-existence in 19 samples of Japanese genome was determined by the LAMP method.

<Amplification by the LAMP Method>

The positions of 5 kinds of primers used in the LAMP method are shown in Table 18. The sequence of each primer is shown below:

F3 primer:
5′-AGCCAGGCTCACTGACG-3′
B3 primer:
5′-CTAGCGGGGCACAGC-3′
FIP primer:
5′-GGTGAAGAAGAGGAAGAGC(F1c)-ACAGGCCGCCGTG(F2)-3′
BIP primer:
5′-TCTCGGTGCCCAC(B1c)-AAAGCTCATAGGGGGATGG(B2)-3′
FLc primer:
5′-ATGCGGGCCAGGGG-3′

60 ng of genome was added to 25 μl reaction solution and reacted at 63° C. for 1 hour. Table 4 shows the composition of the LAMP reaction solution.

TABLE 4
<LAMP primers>

| Primer | Sequence |
|---|---|
| F3 | AGCCAGGCTCACTGACG |
| B3 | CTAGCGGGGCACAGC |
| FIP | GGTGAAGAAGAGGAAGAGC(F1c)-ACAGGCCGCCGTG(F2) |
| BIP | TCTCGGTGCCCAC(B1C)AAAGCTCATAGGGGGATGG(B2) |
| FLc | ATGCGGGCCAGGGG |

<Composition>

| Bst DNA Polymerase | 2 | μL |
|---|---|---|
| 2 × Buffer | 12.5 | μL |
| Tris·HC1 pH8.0 40 mM | | |
| KC1 20 mM | | |
| MgSO₄ 16 mM | | |
| (NH₄)₂SO₄ 20 mM | | |
| Tween20 0.2% | | |
| Betaine 1.6 M | | |
| dNTP 2.8 mM | | |
| F3 primer (20 μM) | 0.5 | μL |
| B3 primer (20 μM) | 0.5 | μL |
| FIP primer (40 μM) | 2 | μL |
| BIP primer (40 μM) | 2 | μL |
| LFc primer (20 μM) | 2 | μL |
| Human genome (60 ng/μL) | 1 | μL |
| Sterilized ultrapure water | 2.5 | μL |
| Total | 25 | μL |

The reaction solution was subjected to 3% agarose gel electrophoresis to confirm the amplified product. As the negative control, sterilized ultrapure water was added in place of the genome and subjected to the electrophoresis in the same manner.

<Detection Sequence>

The detection sequences of CYP2D6 and CYP2D8 are shown below. The CYP2D6 detection sequence was designed to contain a region from the 117-position to 134-position counted from the initiation base of Exon 9, and similarly, CYP2D8 detection sequence was designed to contain a region from the 117-position to 134-position from the initiation base of Exon 9.

CYP2D6 detection sequence:
ACCAGGAAAGCAAAGACACCATGGTGGCT
CYP2D8 detection sequence:
CCAGAAAGCCGACGACACGAGAGTGG

<Preparation of Probe-Immobilized Electrodes>

The nucleotide sequences of probes are shown below. The nucleotide sequence of the probe is a complementary to the detection sequence.

Negative probe:
GACTATAAACATGCTTTCCGTGGCA
CYP2D6 detection probe:
AGCCACCATGGTGTCTTTGCTTTCCTGGT
CYP2D8 detection probe:
CCACTCTCGTGTCGTCGGCTTTCTGG

The above 3 probes were modified at the 3′-terminal with the SH group. The negative probe contained a sequence completely irreverent to the sequence of CYP2D6 and CYP2D8 genes.

A gold electrode was used. The probe was immobilized on the gold electrode by strong binding between thiol at the 3′-terminal of the probe and gold. A solution containing the probe was spotted on a gold electrode, then left for 1 hour, dipped in 1 mM mercaptohexanol solution and washed with 0.2×SSC solution. Each probe was spotted on 4 electrodes. The substrate was washed, then washed with ultrapure water and air-dried, to give a probe-immobilized electrode substrate.

Electrode arrangement:
1 to 4 electrodes: negative probe
5 to 8 electrodes: CYP2D6 detection probe
9 to 12 electrodes: CYP2D8 detection probe

<Determination of Amplified Products>

The amounts of the LAMP amplified products were determined by using the prepared probe-immobilized electrode substrate. Only a salt (final concentration 2×SSC) was added to the amplified product to give sample 1. A salt (final concentration 2×SSC) and a block nucleic acids (final concentration 1.25×10¹⁴ copies/ml) were added to the amplified product to give sample 2. The samples 1 and 2 were reacted with probes of the probe-immobilized electrode substrate prepared above and left at 55° C. for 20 minutes. Thereafter, the probe-immobilized electrode substrate was lightly washed. The probe-immobilized electrode substrate was dipped for 10 minutes in a phosphate buffer containing 50 μM Hoechst 33258 as an intercalator, and then the oxidation current response of Hoechst 33258 molecules was determined.

The nucleotide sequences of the block nucleic acids used are shown below. The nucleotide sequence of the block nucleic acid is a sequence complementary to all or a part of the detection sequence.

CYP2D6 block nucleic acid:
CACCATGGTGTCTTTGCTTTCCTG
CYP2D8 block nucleic acid:
ACTCTCGTGTCGTCGGCT

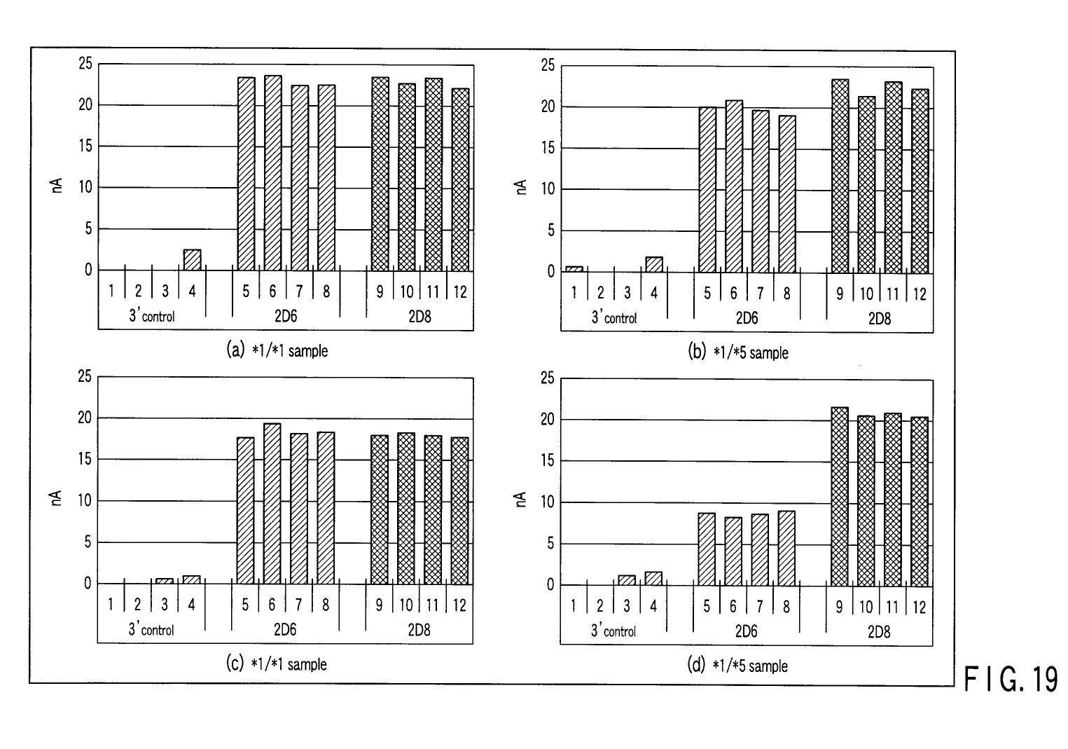

The results are shown in FIG. 19. FIG. 19A is the result of sample 1 (*1/*1 sample), and FIG. 19B is the result of sample 1 (*1/*5 sample). The sample 1 to which the block nucleic acid had not been added did not cause a difference between the signal pattern of *1/*1 sample and the signal pattern of *1/*5 sample. Accordingly, the sample of CYP2D6 whose gene number is 2 could not be distinguished from the sample of CYP2D6 whose gene number is 1.

FIG. 19C is the result of sample 2 (*1/*1 sample), and FIG. 19D is the result of sample 2 (*1/*5 sample). The sample 2 to which the block nucleic acids had been added caused a difference between the signal pattern of *1/*1 sample and the signal pattern of *1/*5 sample. From this result, it was revealed that CYP2D6 whose gene number is 2 could be clearly distinguished from CYP2D6 whose gene number is 1. The result demonstrates that the block nucleic acids are effective.

<Detection Results of the 19 Samples>

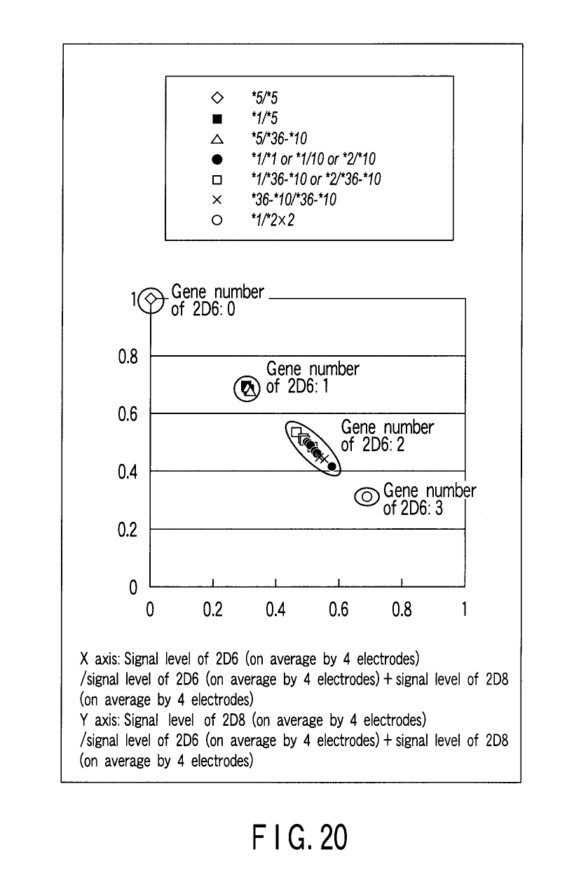

Under the conditions where the block nucleic acids were added, 19 Japanese samples were detected. The results are shown in FIG. 20. As shown in the figure, the positions of the samples whose gene numbers are 0, 1, 2 and 3 respectively were clearly separated from one another. The sample not carrying *36, whose gene number is 2, and the sample carrying two *36, whose gene number is 2, were located almost the same position. From this result, it was revealed that the gene number can be accurately detected without counting *36 as the gene number.

The method of the present invention, as compared with the Southern blot method, can detect a defect and multi-existence in the CYP2D6 gene easily in a short time. Furthermore, the mutation in the CYP2D6 gene can be separately examined by the existing method such as the Taq-man method, invader method, SSCP method, PCR-RFLP, allele specific primer PCR method or allele specific oligonucleotide hybridization analysis, whereby the drug metabolic activity of an individual can be analyzed in detail.

Additional advantages and modifications will readily occur to those skilled in the art. Therefore, the invention in its broader aspects is not limited to the specific details and representative embodiments shown and described herein.

What is claimed is:

1. A method of detecting a defect or multi-existence of a CYP2D6 gene, the method comprising:
a step of amplifying a CYP2D6 gene and a CYP2D8 gene with a pair of primers to obtain amplified products;
a step of determination of amplified products of the CYP2D6 gene and CYP2D8 gene respectively; and
a step of comparing the amount of the amplified product of the CYP2D6 gene with the amount of the amplified product of the CYP2D8 gene,
wherein one of the pair of primers comprises a complementary sequence to a sequence which is common between the CYP2D6 gene and CYP2D8 gene but different from a CYP2D7 gene and which contains a part or all of bases at the 86-, 90- and 93-positions in Exon 9 region of the CYP2D6 gene.

2. The method according to claim 1, wherein the other primer comprises a complementary sequence to a sequence which is common between the CYP2D6 gene and CYP2D8 gene and which contains a sequence upstream of the 180-position in Exon 9 region of the CYP2D6 gene.

3. The method according to claim 1, wherein the amplified products are determined by detecting detection sequences that are specific respectively to the amplified product of the CYP2D6 gene and the amplified product of the CYP2D8 gene.

4. The method according to claim 3, wherein the detection sequences comprise a part or all of a sequence of from a base at the 117-position to a base at the 134-position in Exon 9 regions of the CYP2D6 gene and CYP2D8 gene, respectively.

5. The method according to claim 3, wherein the amplified products are detected with nucleic acid probes complementary to the detection sequences.

6. The method according to claim 5, wherein the nucleic acid probes are immobilized on a substrate.

7. A kit comprising the pair of primers for use in the method according to claim 1.

8. A method of detecting a defect or multi-existence of a CYP2D6 gene, the method comprising:
a step of amplifying a CYP2D6 gene and a CYP2D8 gene by LAMP method with primers to obtain amplified products;
a step of determination of amplified products of the CYP2D6 gene and CYP2D8 gene respectively; and
a step of comparing the amount of the amplified product of the CYP2D6 gene with the amount of the amplified product of the CYP2D8 gene,
wherein one of the primers comprises
a part comprising a complementary sequence to a sequence which is common between the CYP2D6 gene and CYP2D8 gene but different from a CYP2D7 gene and which contains a part or all of bases at the 86-, 90- and 93-positions in Exon 9 region of the CYP2D6 gene; and
a part comprising a complementary sequence to a sequence which is common between the CYP2D6 gene and CYP2D8 gene and which contains a sequence upstream of the 180-position in Exon 9 region of the CYP2D6 gene.

9. The method according to claim 8, wherein the amplified products are determined by detecting detection sequences that are specific respectively to the amplified product of the CYP2D6 gene and the amplified product of the CYP2D8 gene.

10. The method according to claim 9, wherein the detection sequences comprise a part or all of a sequence of from a base at the 117-position to a base at the 134-position in Exon 9 regions of the CYP2D6 gene and CYP2D8 gene, respectively.

11. The method according to claim 9, wherein the amplified products are detected with nucleic acid probes complementary to the detection sequences.

12. The method according to claim 11, wherein the nucleic acid probes are immobilized on a substrate.

13. The method according to claim 8, wherein block nucleic acids are mixed with the amplified products in the step of determination of amplified products.

14. A kit comprising primers for use in the method according to claim 8.

15. The kit for use in the method according to claim 12, comprising primers for the LAMP method and the nucleic acid probes.

16. A probe-immobilized substrate for use in the method of detecting a defect or multi-existence of a CYP2D6 gene, comprising a substrate and the nucleic acid probes according to claim 11, wherein the nucleic acid probes are immobilized on the substrate.

17. The method according to claim 8, wherein one of the primers comprises a part comprising a sequence of SEQ ID No. 13 or a sequence complementary thereto, and a part comprising a sequence of SEQ ID No. 14 or a sequence complementary thereto.

18. The method according to claim 11, wherein the nucleic acid probes include the nucleic acid probe comprising a sequence of SEQ ID No. 19 or a sequence complementary thereto, and the nucleic acid probe comprising a sequence of SEQ ID No. 10 or a sequence complementary thereto.

19. The method according to claim 13, wherein the block nucleic acids include the block nucleic acid comprising a sequence of SEQ ID No. 21 or a sequence complementary thereto, and the block nucleic acid comprising a sequence of SEQ ID No. 22 or a sequence complementary thereto.


SEQUENCE LISTING (29 sequences)

SEQ ID NO: 1 - DNA, 18 bp, Artificial Sequence
  Description of Artificial Sequence Synthetic primer
accaggcccctccaccgg

SEQ ID NO: 2 - DNA, 20 bp, Artificial Sequence
  Description of Artificial Sequence Synthetic primer
tctggtaggggagcctcagc

SEQ ID NO: 3 - DNA, 18 bp, Artificial Sequence
  Description of Artificial Sequence Synthetic primer
accaggcccctccaccgg

SEQ ID NO: 4 - DNA, 19 bp, Artificial Sequence
  Description of Artificial Sequence Synthetic primer
gtggtggggcatcctcagg

SEQ ID NO: 5 - DNA, 23 bp, Artificial Sequence
  Description of Artificial Sequence Synthetic primer
gcagcttcaatgatgagaacctg

SEQ ID NO: 6 - DNA, 21 bp, Artificial Sequence
  Description of Artificial Sequence Synthetic primer
gggtgtcccagcaaagttcat

SEQ ID NO: 7 - DNA, 21 bp, Artificial Sequence
  Description of Artificial Sequence Synthetic primer
gggtgtcccagcaaagttcat

SEQ ID NO: 8 - DNA, 24 bp, Artificial Sequence
  Description of Artificial Sequence Synthetic primer
agagttgggtcagtgggggacatg

SEQ ID NO: 9 - DNA, 17 bp, Artificial Sequence
  Description of Artificial Sequence Synthetic primer
agccaggctcactgacg

SEQ ID NO: 10 - DNA, 15 bp, Artificial Sequence
  Description of Artificial Sequence Synthetic primer
ctagcggggcacagc

SEQ ID NO: 11 - DNA, 19 bp, Artificial Sequence
  Description of Artificial Sequence Synthetic primer
ggtgaagaagaggaagagc

SEQ ID NO: 12 - DNA, 13 bp, Artificial Sequence
  Description of Artificial Sequence Synthetic primer
acaggccgccgtg

SEQ ID NO: 13 - DNA, 13 bp, Artificial Sequence
  Description of Artificial Sequence Synthetic primer
tctcggtgcccac

SEQ ID NO: 14 - DNA, 19 bp, Artificial Sequence
  Description of Artificial Sequence Synthetic primer
aaagctcatagggggatgg

SEQ ID NO: 15 - DNA, 14 bp, Artificial Sequence
  Description of Artificial Sequence Synthetic primer
atgcgggccagggg

SEQ ID NO: 16 - DNA, 29 bp, Artificial Sequence
  Description of Artificial Sequence Synthetic oligonucleotide
accaggaaagcaaagacaccatggtggct

SEQ ID NO: 17 - DNA, 26 bp, Artificial Sequence
  Description of Artificial Sequence Synthetic oligonucleotide
ccagaaagccgacgacacgagagtgg

SEQ ID NO: 18 - DNA, 25 bp, Artificial Sequence
  Description of Artificial Sequence Synthetic probe
gactataaacatgctttccgtggca

SEQ ID NO: 19 - DNA, 29 bp, Artificial Sequence
  Description of Artificial Sequence Synthetic probe
agccaccatggtgtctttgctttcctggt

SEQ ID NO: 20 - DNA, 26 bp, Artificial Sequence
  Description of Artificial Sequence Synthetic probe
ccactctcgtgtcgtcggctttctgg

SEQ ID NO: 21 - DNA, 24 bp, Artificial Sequence
  Description of Artificial Sequence Synthetic oligonucleotide
caccatggtgtctttgctttcctg

SEQ ID NO: 22 - DNA, 18 bp, Artificial Sequence
  Description of Artificial Sequence Synthetic oligonucleotide
actctcgtgtcgtcggct

SEQ ID NO: 23 - DNA, 252 bp, Homo sapiens
gccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttcc

SEQ ID NO: 24 - DNA, 252 bp, Homo sapiens
gccgccgtgcatgcctcggggagcccctggcccgcatggagctcttcctcttcttcacctccctgctgcagcacttcagcttctccgtggccgccggacagccccggcccagccactctcgtgtcgtcagctttctggtgaccccatccccctatgagctttgtgctgtgccccgctagaatggggtacctagtccccagcctgttccctagccagaggctctaatgtacaataaagcaatgtggtagttcc

SEQ ID NO: 25 - DNA, 252 bp, Homo sapiens
gccgccgtgcatgcctcggggagcccctggcccgcatggagctcttcctcttcttcacctccctgctgcagcacttcagcttctccgtggccgccggacagccccggcccagccactctcgtgtcgtcagctttctggtgaccccatccccctatgagctttgtgctgtgccccgctagaatggggtacctagtccccagcctgctccctagccagaggctctaatgtacaataaagcaatgtggtagttcc

SEQ ID NO: 26 - DNA, 300 bp, Homo sapiens
ggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagca

SEQ ID NO: 27 - DNA, 300 bp, Homo sapiens
ggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctccgtggccgccggacagccccggcccagccactctcgtgtcgtcagctttctggtgaccccatccccctatgagctttgtgctgtgccccgctagaatggggtacctagtccccagcctgttccctagccagaggctctaatgtacaataaagca

SEQ ID NO: 28 - DNA, 298 bp, Homo sapiens
ggagtcttgcaggggtatcacccgggagccaggctcactgacgccctcccctccccacaggccgccgtgcatgcctcggggagcccctggcccgcatagagctcttcctcttcttcacctccctgctgcagcacttcagcttctcggtgcccaccggacagccccggcccagccactctcgtgtcgtcggctttctggtgacgccatccccctatgagctttgtgctgtgccccgctagagttgctcctcagctgggaccctgttgtacaataaattagtctagtggctcccacttgg

SEQ ID NO: 29 - DNA, 20 bp, Artificial Sequence
  Description of Artificial Sequence Synthetic primer
ccatggtgtctttgctttcc